(19) United States
(12) Patent Application Publication
(10) Pub. No.: US 2017/0209127 A1
(43) Pub. Date: Jul. 27, 2017

(54) WIRELESS CHARGING SYSTEM FOR WIRELESSLY CHARGING ULTRASOUND IMAGING SYSTEM

(71) Applicant: General Electric Company, Schenectady, NY (US)

(72) Inventors: Arun Kumar Raghunathan, Bangalore (IN); Sigmund Frigstad, Trondheim (NO); Adnan Bohori, Bangalore (IN); Srinivas Varna, Bangalore (IN); Viswanathan Kanakasabai, Bangalore (IN); Suma Memana Narayana Bhat, Bangalore (IN)

(21) Appl. No.: 15/328,540

(22) PCT Filed: Jul. 28, 2015
(86) PCT No.: PCT/US2015/042384

§ 371 Date: Jan. 24, 2017

(30) Foreign Application Priority Data
Jul. 30, 2014 (IN) 3701/CHE/2014

(51) Int. Cl. A61B 8/00; H02J 7/02; H02J 50/10; G01S 7/52

(52) U.S. Cl. CPC A61B 8/56; A61B 8/4427; A61B 8/4433; A61B 8/4472; G01S 7/52096; H02J 7/025; H02J 50/10

(57) ABSTRACT

A wireless charging system for wirelessly charging an ultrasound imaging system is disclosed. The wireless charging system comprise one or more primary coils connected to a power source and is capable of transmitting power from the power source. The primary coil of the one or more primary coils is disposed in a charging unit of the ultrasound imaging system. One or more secondary coils are configured to receive power transmitted from the primary coil. One or more field focusing elements are positioned between the primary coil and the secondary coil. A field focusing element is capable of focusing the magnetic field from the primary coil onto the secondary coil for wirelessly transferring power to one or more of the ultrasound device and the probe of the ultrasound imaging system.

### FIELD OF THE INVENTION

The subject matter disclosed herein relates to charging of ultrasound imaging system. More specifically the subject matter relates to wirelessly charging a probe and an ultrasound device.

### BACKGROUND OF THE INVENTION

Ultrasound imaging is one of the commonly used diagnosing methods for analyzing a medical condition of a patient. An ultrasound imaging apparatus includes an ultrasound probe built-in with a transducer array and an apparatus connected to the ultrasound probe. Ultrasonic waves are transmitted towards the subject from the ultrasound probe. Thereafter the ultrasound probe receives ultrasonic echoes from the subject and generates an ultrasound image by electrically processing these ultrasonic echoes. Recently to eliminate the issues associated with usage of communication cables connecting the ultrasound probe with the ultrasound apparatus, ultrasound probes having wireless capability have been introduced. In this scenario the ultrasound probe needs to be powered and hence rechargeable batteries are provided. To recharge these batteries power is supplied from the ultrasound apparatus or an ultrasound docking or charging device is provided where the probe can be connected or docked for charging their batteries. Once the ultrasound probe runs out of charge then the probe needs be docked in which may be render it inconvenient for the user. Moreover multiple times the probe need to be carried to the docking device for charging based on usage. Alternatively the batteries may need to be replaced with recharged batteries time and time again. Some instances the probe need to be used for long duration scans and thus bulkier batteries need to be used which makes the probe altogether more bulky. So handling bulky probes may be cumbersome and also affects the comfort level of the user for a long duration scan.

Accordingly, a need exists for a system for wirelessly charging the probe and the ultrasound device.

### SUMMARY OF THE INVENTION

The object of the invention is to provide a system for wirelessly charging a probe and an ultrasound device, which overcomes one or more drawbacks of the prior art. This is achieved by a wireless charging system that can be used to wirelessly transfer power to the probe and the ultrasound device as defined in the independent claim.

One advantage with the disclosed system is that it can wirelessly charge the probe and the ultrasound device from a considerable distance which renders it convenient for the user to carry the probe and the ultrasound device for performing ultrasound imaging. In an instance if the ultrasound probe is remote from a charging device or a docking device the wireless charging system can power the ultrasound probe so that it can be used continuously for performing ultrasound imaging.

In an embodiment a wireless charging system for wirelessly charging an ultrasound imaging system is disclosed. The wireless charging system comprise one or more primary coils connected to a power source and is capable of transmitting power from the power source. The primary coil of the one or more primary coils is disposed in a charging unit of the ultrasound imaging system. One or more secondary coils are configured to receive power transmitted from the primary coil. One or more field focusing elements are positioned between the primary coil and the secondary coil. A field focusing element is capable of focusing the magnetic field from the primary coil onto the secondary coil for wirelessly transferring power to one or more of the ultrasound device and the probe of the ultrasound imaging system.

In another embodiment an ultrasound imaging system configured to receive power wirelessly is disclosed. The ultrasound imaging system includes an ultrasound device, a probe and a charging unit. A wireless charging system is provided that includes one or more primary coils connected to a power source and is capable of transmitting power from the power source, wherein the primary coil of the one or more primary coils is disposed in the charging unit. One or more secondary coils are configured to receive the power transmitted from the primary coil. One or more field focusing elements are positioned between the primary coil and the secondary coil. A field focusing element is positioned between the primary coil and the secondary coil and capable of focusing the magnetic field from the primary coil onto the secondary coil for wirelessly transferring power to one or more of the ultrasound device and the probe.

In yet another embodiment an ultrasound imaging system configured to receive power wirelessly is disclosed. The ultrasound imaging system includes an ultrasound device, a charging unit and a probe. The ultrasound imaging system includes a wireless charging system comprising one or more primary coils connected to a power source and is capable of transmitting power from the power source. A primary coil of the one or more primary coils and the power source are communicably connected to the charging unit. A plurality of secondary coils is configured to receive the power transmitted from the primary coil. At least two secondary coils of the plurality of coils are disposed in the probe. A secondary coil is orthogonally arranged with respect to another secondary coil in the probe. One or more field focusing elements are positioned between the primary coil and the secondary coil, wherein a field focusing element of the one or more field focusing elements is capable of focusing the magnetic field from the primary coil onto the secondary coil for wirelessly transferring power to one or more of the ultrasound device and the probe.

A more complete understanding of the present invention, as well as further features and advantages thereof, will be obtained by reference to the following detailed description and drawings.

### BRIEF DESCRIPTION OF THE DRAWINGS

FIG. 1 is a schematic illustration of an exemplary wireless charging system in accordance with an embodiment;

FIG. 2 is a schematic illustration of an exemplary embodiment of the field focusing element including a plurality of resonators arranged in an array for focusing a magnetic field from the primary coil to the secondary coil accordance with an embodiment;

FIG. 3 is a schematic illustration of an ultrasound imaging system embodied with a wireless charging system for transferring power to an ultrasound probe and an ultrasound device in accordance to an embodiment;

FIG. 4 is a schematic illustration showing wireless transfer of power to the ultrasound device from the charging unit according to an exemplary embodiment;

FIG. 5 is a schematic illustration showing wireless transfer of power to the ultrasound probe from the charging unit according to an exemplary embodiment; and

FIG. 6 is a schematic illustration showing wireless transfer of power to the ultrasound probe from the ultrasound device according to an exemplary embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical and other changes may be made without departing from the scope of the embodiments. The following detailed description is, therefore, not to be taken as limiting the scope of the invention.

As discussed in detail below, embodiments of a wireless charging system for wirelessly charging an ultrasound imaging system is disclosed. The wireless charging system comprise one or more primary coils connected to a power source and is capable of transmitting power from the power source. The primary coil of the one or more primary coils is disposed in a charging unit of the ultrasound imaging system. One or more secondary coils are configured to receive power transmitted from the primary coil. One or more field focusing elements are positioned between the primary coil and the secondary coil. A field focusing element is capable of focusing the magnetic field from the primary coil onto the secondary coil for wirelessly transferring power to one or more of the ultrasound device and the probe of the ultrasound imaging system.

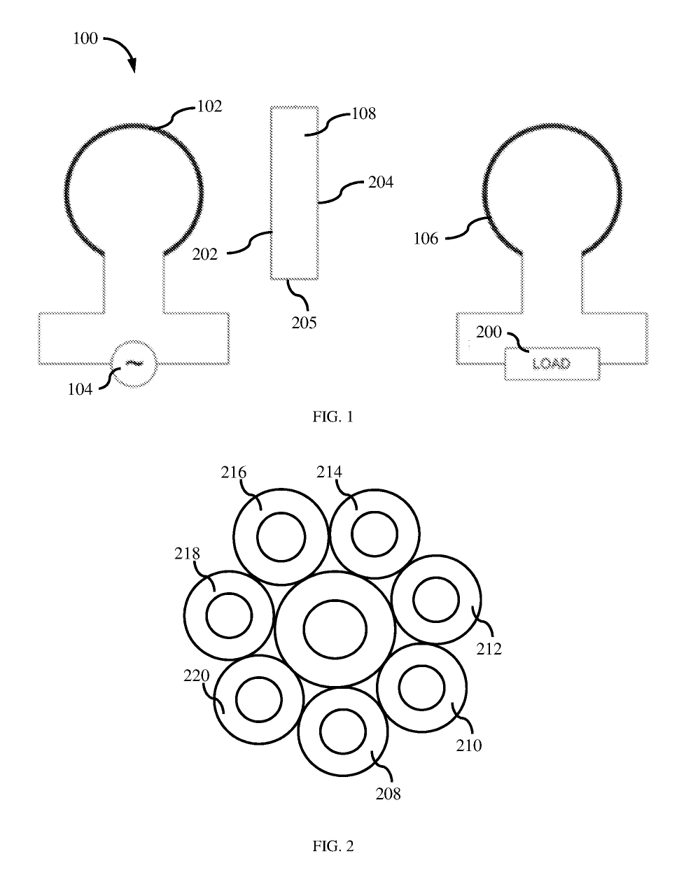

FIG. 1 illustrates an exemplary contactless power transfer system 100 (i.e. a wireless charging system) according to an embodiment of the invention including a primary coil 102 coupled to a power source 104 and configured to produce a magnetic field (not shown). A secondary coil 106 is configured to receive power from the primary coil 102. A field focusing element 108 is disposed between the primary coil 102 and the secondary coil 106 for focusing the magnetic field from power source 104. In another embodiment, the field focusing element may be used to focus electro-magnetic fields. The terms “magnetic field focusing element” and “field focusing element” are used interchangeably. In one embodiment, the magnetic field focusing element 108 is configured as a self-resonant coil and has a standing wave current distribution when excited via the primary coil. In another embodiment, the magnetic field focusing element 108 is configured as a sub wavelength resonator. In yet another embodiment, the magnetic field focusing element includes multiple resonators operating as an active array or a passive array and each resonator configured as a self-resonant coil with a standing wave current distribution. In yet another embodiment, the magnetic field focusing element includes multiple sets of such resonators, each such resonator set excited at a particular phase. It may be appreciated that, when exciting the sets of resonators via different phases, field focusing may be enhanced in a desired direction.

The magnetic field focusing element 108 is further configured to focus the magnetic field onto the secondary coil 106 enhancing the coupling between the primary coil 102 and the secondary coil 106. In the illustrated embodiment, the field focusing element 108 is placed closer to the primary coil 102 as an example. It may be advantageous in certain systems to place the field focusing element 108 closer to the secondary coil 106. A load 200 is coupled to the secondary coil 106 to utilize the power transferred from the power source 104. In certain embodiments, the contactless power transfer system 100 may also be configured to simultaneously transfer power from the secondary coil 106 to the primary coil 102 such that the system is capable of bidirectional power transfer. Non-limiting examples of potential loads include a bulb, a battery, a computer, a sensor, or any device that requires electrical power for operation.

The contactless power transfer system 100 may be used to transfer power from the power source 104 to the load 200. In one embodiment, the power source 104 comprises a single phase AC power generator or three phase AC power generator or a DC power generator in combination with power conversion electronics to convert the power to a higher frequency. When the primary coil 102 is excited at the resonant frequency of the magnetic field focusing element 108, a standing wave current distribution is developed within the magnetic field focusing element 108 between two open ends (202, 204) of the field focusing element. The standing wave current distribution leads to a non-uniform magnetic field distribution around the magnetic field focusing element 108. Such non-uniform current distribution is configured to focus magnetic field in any desired direction, such as, in a direction of the secondary coil 106 in this example. When operating at resonant frequency, even a small excitation to the magnetic field focusing element 108 produces large amplitude of current distribution along the length 205 of the magnetic field focusing element 108. Large current magnitude of non-uniform distribution leads to an amplified and focused magnetic field in the direction of secondary coil 106 that result in higher efficiency of power transfer.

FIG. 2 is a schematic illustration of an exemplary embodiment of the field focusing element 108 including a plurality of resonators arranged in an array for focusing a magnetic field from the primary coil 102 to the secondary coil 106. The plurality of resonators including a resonator 208, a resonator 210, a resonator 212, a resonator 214, a resonator 216, a resonator 218 and a resonator 220 are configured to act as a single unit wherein a resultant magnetic field is induced by the respective magnetic fields of the plurality of resonators in the array by interfering constructively (adding) in a desired direction to achieve magnetic field focusing and interfering destructively (canceling each other) in remaining space. Although an embodiment of the array is shown, there may be various other forms of array that can be formed from the plurality of resonators. The resultant magnetic field is transmitted to the secondary coil 106 that is electronically coupled to the load (refer to FIG. 1). Moreover, in a particular embodiment, the at least one resonator includes at least two different resonant frequencies. For example, one resonator (for example the resonator 208) may include two different resonant frequencies or two resonators (such as the resonator 210 and the resonator 212) which may each include a different resonant frequency. In a more specific embodiment, having at least two different resonant frequencies enable transfer of power and data signals simultaneously.

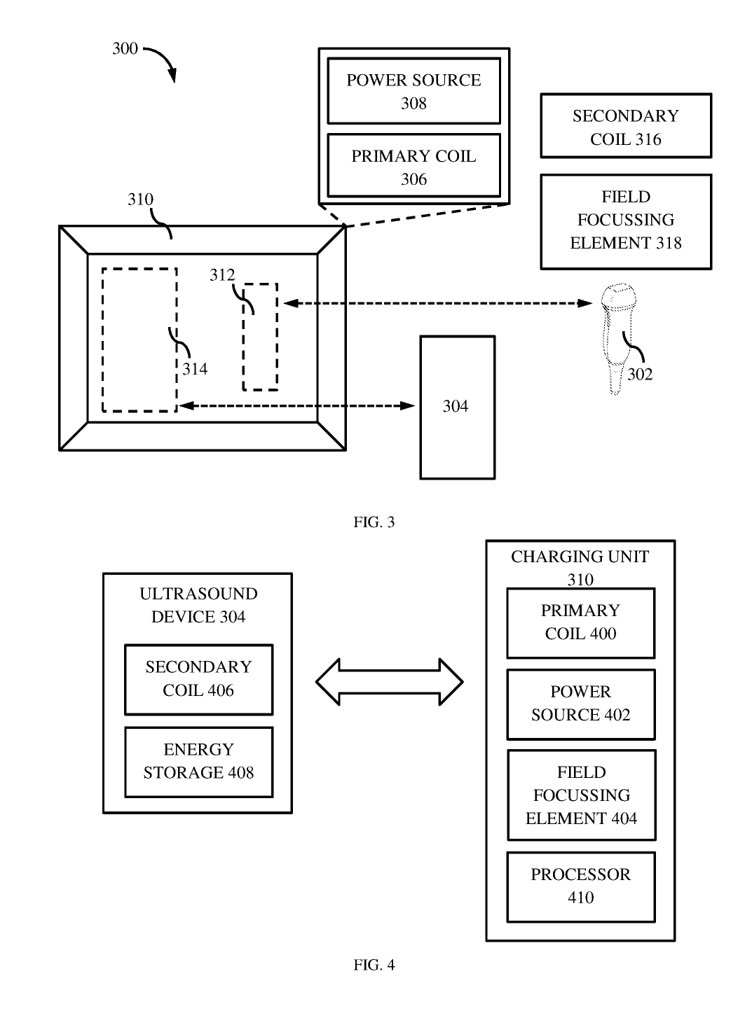

FIG. 3 is a schematic illustration of an ultrasound imaging system 300 embodied with a wireless charging system for transferring power to an ultrasound probe 302 and an ultrasound device 304 in accordance to an embodiment. The wireless charging system includes a primary coil 306 connected to a power source 308. The primary coil 306 and the power source 308 are present in a charging unit 310. The power source 308 enables the magnetic field to be generated at the primary coil 306. In an embodiment the charging unit 310 acts as a docking unit where the probe 302 and the ultrasound device 304 can be docked for charging them. The probe 302 and the ultrasound device 304 can be docked in their appropriate docking slots such as a docking slot 312 and a docking slot 314 respectively for charging. In another embodiment the charging unit 310 may be in the form of a charging pad on which the probe 302 and the ultrasound device 304 can be placed for charging or powering. Even though only few embodiments of the charging unit 310 are described herein it may be appreciated that other embodiments may be present wherein the charging unit may have different physical and functional configurations without deviating from the scope of this disclosure.

The magnetic field is focused on a secondary coil 316 for transferring power to one of the probe 302 and the ultrasound device 304. A field focusing element 318 focusses the magnetic field onto the secondary coil 316 for charging one of the probe 302 and the ultrasound device 304. It may be noted that only one primary coil, secondary coil and field focusing element are shown in the FIG. 3 for sake of convenience of representation, however it may be envisioned that multiple primary coils, secondary coils and field focusing elements may be disposed in the charging unit, the probe 302 and the ultrasound device 304 for performing wireless transfer of power without deviating from the scope of this disclosure. In an embodiment the probe 302 may include two secondary coils that may be orthogonally arranged with respect to each other. The magnetic field is focused onto these two secondary coils for transferring the power to the probe 302. It may be envisioned that an orthogonal arrangement of the secondary coils is according to an embodiment and other embodiments may have different arrangements of the secondary coils without deviating from the scope of this disclosure.

In a scenario the probe 302 may be at a position closer to the charging unit 310. Here the probe 302 may be charged directly by the charging unit 310 through the primary coil 306 without the need of the field focusing element 318. Another instance may have the probe 302 placed in a probe holder (not shown in FIG. 3) of an ultrasound device. The probe holder may have one or more primary coils that transfer power to the probe 302. The power may be focused on to the secondary coils in the probe 302 by the field focusing element 318. The process of transferring power may be more efficient here as the probe 302 is positioned very close to the source of the power i.e. the primary coils in the probe holder.

FIG. 4 is a schematic illustration showing wireless transfer of power to the ultrasound device 304 from the charging unit 310 according to an exemplary embodiment. The ultrasound device 304 may be positioned proximate to the charging unit 310. In an embodiment the ultrasound device 304 may be a mobile device or a portable device having an ultrasound application for performing the ultrasound image processing. In other embodiments the ultrasound device 304 may be usual hardware device for performing the ultrasound scanning operations having a user interface and a display unit. The user interface may include touch pads and interactive switches or elements for performing the ultrasound scanning operations. The charging unit 310 includes a primary coil 400, a power source 402 and a field focusing element 404. The power source 402 facilitates generation of magnetic field at the primary coil 400 which is transmitted to a secondary coil 406 in the ultrasound device 304. The field focusing element 404 focuses the magnetic field from the primary coil 400 onto the secondary coil 406.

The magnetic field focused on the secondary coil 406 may be used to generate power or transfer power into energy storage 408 of the ultrasound device 304. The energy storage 408 may be a rechargeable battery. In another embodiment the energy storage 408 may be a capacitive based storage for example an ultra-capacitor, a super capacitor and so on. During operation in a hospital environment the ultrasound device 304 is used for performing ultrasound imaging on the patient. The ultrasound device 304 may be connected to an ultrasound probe (not shown in FIG. 4) for sending ultrasound signals onto the patient's body and obtaining appropriate ultrasound images. The charging unit 310 may be also present in the same location where the ultrasound imaging or scanning is performed on the patient. As the charging unit 310 identifies that the ultrasound device 304 is within its vicinity and can communicate the charging unit 310 establishes communication with the ultrasound device 304. Here the charging unit 310 starts transferring power to the ultrasound device 304 so as to charge or energize the energy storage 408. The energy storage 408 stores the power facilitating the functioning of the ultrasound device 304. In an embodiment the transfer of power occurs only upon confirmation by a user of the ultrasound device 304. For instance the user may need to provide a confirmation or trigger from the ultrasound device 304 for the transfer of power to the energy storage 408 to commence. Thus the ultrasound device 304 can also be charged when in use and need not be in a docked position in the charging unit 310.

In yet another embodiment the charging unit 310 may be configured to power the ultrasound device 304. The process of powering involves sending small packets of charge. The need for powering may arise in an exemplary scenario when the ultrasound device 304 is performing scanning and power runs out from the device. In such situations the charging unit 310 may send small packets of charge to power the ultrasound device 304 for completing the scanning procedure. The small packets of charge may not be stored in the energy storage 408 and thus may be consumed for completing the scanning procedure.

Further the secondary coil 406 may be configured to transfer exchange data from the ultrasound device 304 to the charging unit 310. The exchange data may include but are not limited to, status of the energy storage 408, capability history of the energy storage 408, status of the ultrasound device 304, and charging data associated with the energy storage 408. The exchange data may be received by the primary coil 400. The exchange data may be stored in a memory (not shown in FIG. 4) of the charging unit 310. The exchange data may be processed by a processor 410 to determine when charging unit 310 needs to transfer power to the ultrasound device 304. In an embodiment based on the exchange data the charging unit 310 may also determine when power needs to be transferred for storing the energy storage 408 and when the ultrasound device 304 needs to be powered by sending small packets of charge.

In an embodiment the primary coil 400, the power source 402 and the field focusing element 404 may be configured as a single unit that can be disposed in the charging unit 310. Further the single unit may be a pluggable type module that can be inserted or communicably connected to the charging unit 310. Even though only few alternative embodiments of the primary coil 400, the power source 402 and the field focusing element 404 forming a single unit is described it may be envisioned that alternative arrangements of these components may be possible within scope of this disclosure.

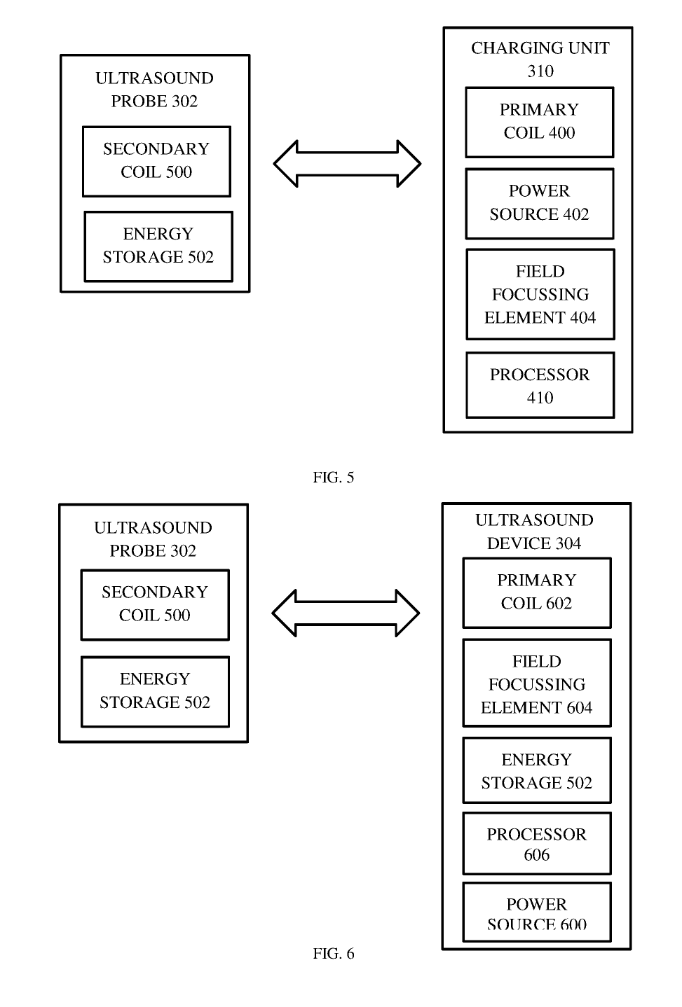

FIG. 5 is a schematic illustration showing wireless transfer of power to the ultrasound probe 302 from the charging unit 310 according to an exemplary embodiment. The ultrasound probe 302 may be positioned proximate to the charging unit 310. The ultrasound probe 302 may be communicably connected to the ultrasound device 304. The connection between the probe 302 and the ultrasound device 304 may be a wired or wireless connection. In an embodiment the probe 302 and the ultrasound device 204 may communicate over but not limited to, a Bluetooth® connection, a Wi-Fi connection and so on. Thus the probe 302 may be wireless or wired probe. As described earlier in conjunction with FIG. 4, the power source 402 facilitates generation of magnetic field at the primary coil 400 which is transmitted to a secondary coil 406 in the ultrasound device 304. The field focusing element 404 focuses the magnetic field from the primary coil 400 on to a secondary coil 500.

The magnetic field focused on the secondary coil 500 may be used to generate power or transfer power into energy storage 502 of the ultrasound probe 302. The energy storage 502 may be a rechargeable battery. In another embodiment the energy storage 502 may be a capacitive based storage for example an ultra-capacitor, a super capacitor and so on. During operation in a hospital environment the ultrasound probe 302 is used for performing ultrasound imaging on the patient. The ultrasound probe 302 sends ultrasound signals onto the patient's body and obtains image data to generate appropriate ultrasound images. The charging unit 310 may be also present in the same location where the ultrasound imaging or scanning is performed on the patient. As the charging unit 310 identifies that the ultrasound probe 302 is within its vicinity and can communicate, the charging unit 310 establishes communication with the ultrasound probe 302. Here the charging unit 310 starts transferring power to the ultrasound probe 302 so as to charge or energize the energy storage 502. The energy storage 502 stores the power facilitating the functioning of the ultrasound probe 302. In an embodiment the transfer of power occurs only upon confirmation by a user of the ultrasound probe 302. For instance the user may need to provide a confirmation or trigger from the ultrasound probe 302 for the transfer of power to the energy storage 502 to commence. Thus the ultrasound probe 302 can also be charged when in use and need not be in a docked position in the charging unit 310. Hence the probe 302 can be conveniently carried during the ultrasound scanning procedure. When the ultrasound probe 302 is more remote or far away from the charging unit 310 the energy storage 502 can provide adequate power for its functioning without interrupting the scanning procedure.

In yet another embodiment the charging unit 310 may be configured to power the ultrasound probe 302. The process of powering involves sending small packets of charge. The need for powering may arise in an exemplary scenario when the ultrasound probe 302 is performing scanning and power runs out from the probe. In such situations the charging unit 310 may send small packets of charge to power the ultrasound probe 302 for completing the scanning procedure. The small packets of charge may not be stored in the energy storage 502 and thus may be consumed for completing the scanning procedure.

Further the secondary coil 500 may be configured to transfer exchange data from the ultrasound probe 302 to the charging unit 310. The exchange data may include but are not limited to, status of the energy storage 502, capability history of the energy storage 502, status of the ultrasound probe 302, and charging data associated with the energy storage 502. The exchange data may be received by the primary coil 400. The exchange data may be stored in a memory (not shown in FIG. 4) of the charging unit 310. The exchange data may be processed by a processor 410 to determine when the charging unit 310 needs to transfer power to the ultrasound probe 302. In an embodiment based on the exchange data the charging unit 310 may also determine when power needs to be transferred for storing in the energy storage 502 and when the ultrasound probe 302 needs to be powered by sending small packets of charge.

As discussed in FIG. 4 and FIG. 5 the ultrasound probe 302 and the ultrasound device 304 powered or charged remotely which makes it convenient for the user to perform ultrasound imaging on the patient.

As described earlier the ultrasound probe 302 is in communication with the ultrasound device 304 and thus the ultrasound device 304 may be capable of transferring power to the probe 302. FIG. 6 is a schematic illustration showing wireless transfer of power to the ultrasound probe 302 from the ultrasound device 304 according to an exemplary embodiment. The ultrasound probe 302 may be positioned proximate to the ultrasound device 304. A power source 600 in the ultrasound device 304 facilitates generation of magnetic field at a primary coil 602 which is transmitted to the secondary coil 500 in the ultrasound device 304. A field focusing element 604 focuses the magnetic field from the primary coil 602 on to the secondary coil 500. The magnetic field focused on the secondary coil 500 may be used to generate power or transfer power into the energy storage 502 of the ultrasound probe 302. For instance in the ultrasound probe 302 is used for performing ultrasound imaging on the patient. The ultrasound probe 302 sends ultrasound signals onto the patient's body and obtains image data to generate appropriate ultrasound images. The ultrasound device 304 may be also present in the same location where the ultrasound imaging or scanning is performed. As the ultrasound device 304 identifies that the ultrasound probe 302 is within its vicinity and can communicate, the charging unit 310 establishes communication with the ultrasound probe 302. The ultrasound device 304 starts transferring power to the ultrasound probe 302 so as to charge or energize the energy storage 502. The energy storage 502 stores the power facilitating the functioning of the ultrasound probe 302. In an embodiment the transfer of power occurs only upon confirmation by a user of the ultrasound probe 302. For instance the user may need to provide a confirmation or trigger from the ultrasound probe 302 for the transfer of power to the energy storage 502 to commence. Thus the ultrasound probe 302 can also be charged when in use.

In an embodiment the ultrasound device 304 may be configured to power the ultrasound probe 302. The process of powering involves sending small packets of charge as discussed earlier in conjunction with FIG. 4. The need for powering may arise in an exemplary scenario when the ultrasound probe 302 is performing scanning and power runs out from the probe. In such situations the ultrasound device 310 may send small packets of charge to power the ultrasound probe 302 for completing the scanning procedure. The small packets of charge may not be stored in the energy storage 502 and may be consumed directly for completing the scanning procedure.

Further the secondary coil 500 may be configured to transfer exchange data from the ultrasound probe 302 to the ultrasound device 304. The exchange data may include but are not limited to, status of the energy storage 502, capability history of the energy storage 502, status of the ultrasound probe 302, and charging data associated with the energy storage 502. The exchange data may be received by the primary coil 602. The exchange data may be stored in a memory (not shown in FIG. 4) of the charging unit 310. The exchange data may be processed by a processor 606 to determine when the ultrasound device 304 needs to transfer power to the ultrasound probe 302. In an embodiment based on the exchange data the ultrasound probe 302 may also determine when power needs to be transferred for storing in the energy storage 502 and when the ultrasound probe 302 needs to be powered by sending small packets of charge.

In an exemplary embodiment the primary coil 602 may act as a secondary coil and hence only a single coil may be present to perform the function of both these coils. The processor 606 may be configured to shift the functioning capability of the primary coil 602 to the secondary coil and vice versa depending on the scenarios such as the ultrasound device 310 being provided power from the charging unit 310 and the ultrasound device 310 transferring power to the ultrasound probe 302.

In an embodiment the primary coil 602, the power source 600 and the field focusing element 604 may be configured as a single unit that can be disposed in the ultrasound device 304. Further the single unit may be a pluggable type module that can be inserted or communicably connected to the ultrasound device 304. Even though only few alternative embodiments of the primary coil 602, the power source 600 and the field focusing element 604 forming a single unit is described it may be envisioned that alternative arrangements of these components may be possible within scope of this disclosure.

From the foregoing, it will be appreciated that the above disclosed wireless charging system for wirelessly charging an ultrasound device and an ultrasound probe provides numerous benefits to healthcare enterprises, such as avoiding the need for docking the ultrasound device and/or the probe in a charging unit i.e. a docking unit. The probe and the ultrasound device as they are wirelessly connected and portable the user can move this around and still not be concerned of charging the probe. This is because the probe can be powered or charged by the ultrasound device. Further as the probe and the ultrasound device are wirelessly charged or powered there is no discomfort for the user due to wires or constraints of length of wires. These wired connections may have multiple reliability issues which are avoided and hence more convenient for the user and increases the longevity of the probes.

This written description uses examples to disclose the invention, including the best mode, and also to enable any person skilled in the art to practice the invention, including making and using any computing system or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A wireless charging system for wirelessly charging an ultrasound imaging system, the wireless charging system comprises:
at least one primary coil connected to a power source and is capable of transmitting power from the power source, wherein a primary coil of the at least one primary coil and the power source is communicably connected to a charging unit of the ultrasound imaging system;
at least one secondary coil configured to receive the power transmitted from the primary coil; and
at least one field focusing element positioned between the primary coil and the secondary coil, wherein a field focusing element of the at least one field focusing element is capable of focusing the magnetic field from the primary coil onto the secondary coil for wirelessly transferring power to at least one of a ultrasound device and a probe of the ultrasound imaging system.

2. The wireless charging system of claim 1, wherein a secondary coil of the at least one secondary coil is configured to transfer the power to an energy storage of at least one of the ultrasound device and the probe.

3. The wireless charging system of claim 1, wherein: a secondary coil of the at least one secondary coil is disposed in the ultrasound device; and the field focusing element of the at least one field focusing element is disposed in the charging unit for wirelessly transferring power to the ultrasound device.

4. The wireless charging system of claim 1, wherein a secondary coil of the at least one secondary coil is disposed in the probe.

5. The wireless charging system of claim 4, wherein the field focusing element of the at least one field focusing element is disposed in the charging unit for wirelessly transferring power to the probe.

6. The wireless charging system of claim 4, wherein the charging unit is configured to wirelessly transfer power directly to the probe without using the at least one field focusing element.

7. The wireless charging system of claim 6, wherein a field focusing element of the at least one field focusing element is disposed in the ultrasound device; and wherein a primary coil of the at least one primary coil is disposed in the ultrasound device, wherein the primary coil in the ultrasound device is configured to wirelessly transfer power to the secondary coil in the probe.

8. The wireless charging system of claim 1, wherein a field focusing element of the at least one field focusing element comprises a plurality of resonators having two or more resonant frequencies.

9. The wireless charging system of claim 1, wherein wirelessly transferring power to at least one of the ultrasound device and the probe of the ultrasound imaging system comprises:
wirelessly powering one of the ultrasound device and the probe; and
wirelessly charging an energy storage of one of the ultrasound device and the probe.

10. The wireless charging system of claim 1, wherein the wireless charging system is configured to exchange data between the charging unit and at least one the ultrasound device and the probe.

11. An ultrasound imaging system configured to receive power wirelessly, wherein the ultrasound imaging system have an ultrasound device, a charging unit and a probe, the ultrasound imaging system comprising:
a wireless charging system comprising:
at least one primary coil connected to a power source and is capable of transmitting power from the power source, wherein a primary coil of the at least one primary coil and the power source are communicably connected to the charging unit;
at least one secondary coil configured to receive the power transmitted from the primary coil; and
at least one field focusing element positioned between the primary coil and the secondary coil, wherein a field focusing element of the at least one field focusing element is capable of focusing the magnetic field from the primary coil onto the secondary coil for wirelessly transferring power to at least one of the ultrasound device and the probe.

12. The ultrasound imaging system of claim 11, wherein a secondary coil of the at least one secondary coil is configured to transfer the power to energy storage in at least one of the ultrasound device and the probe.

13. The ultrasound imaging system of claim 11, wherein a secondary coil of the at least one secondary coil is disposed in the ultrasound device; and the field focusing element of the at least one field focusing element is disposed in the charging unit for wirelessly transferring power to the ultrasound device.

14. The ultrasound imaging system of claim 11, wherein a secondary coil of the at least one secondary coil is disposed in the probe.

15. The ultrasound imaging system of claim 14, wherein the field focusing element of the at least one field focusing element is disposed in the charging unit for wirelessly transferring power to the probe.

16. The ultrasound imaging system of claim 15, wherein a field focusing element of the at least one field focusing element is disposed in the ultrasound device; and wherein a primary coil of the at least one primary coil is disposed in the ultrasound device, wherein the primary coil in the ultrasound device is configured to wirelessly transfer power to the secondary coil in the probe.

17. The ultrasound imaging system of claim 14 further comprises a probe holder having the at least one primary coil, wherein the probe is positioned in the probe holder and charged by the at least one primary coil.

18. The ultrasound imaging system of claim 11, wherein wirelessly transferring power to at least one of the ultrasound device and the probe comprises:
wirelessly powering one of the ultrasound device and the probe; and
wirelessly charging an energy storage of one of the ultrasound device and the probe.

19. The ultrasound imaging system of claim 11, wherein the probe is a wireless probe and the ultrasound device is a portable wireless ultrasound device.

20. An ultrasound imaging system configured to receive power wirelessly, wherein the ultrasound imaging system have an ultrasound device, a charging unit and a probe, the ultrasound imaging system comprising:
a wireless charging system comprising:
at least one primary coil connected to a power source and is capable of transmitting power from the power source, wherein a primary coil of the at least one primary coil and the power source are communicably connected to the charging unit;
a plurality of secondary coils configured to receive the power transmitted from the primary coil, wherein at least two secondary coils of the plurality of coils are disposed in the probe, wherein a secondary coil is orthogonally arranged with respect to another secondary coil in the probe; and
at least one field focusing element positioned between the primary coil and the secondary coil, wherein a field focusing element of the at least one field focusing element is capable of focusing the magnetic field from the primary coil onto the secondary coil for wirelessly transferring power to at least one of the ultrasound device and the probe.
